(19) United States
(12) Patent Application Publication
(10) Pub. No.: US 2004/0018524 A1
(43) Pub. Date: Jan. 29, 2004

(54) Method of screening for genes that influence pathological conditions or survival of animals infected with pathogen

(76) Inventor: Junichi Watanabe, Tokyo (JP)

Correspondence Address: KLARQUIST SPARKMAN, LLP, 121 SW SALMON STREET, SUITE 1600, PORTLAND, OR, 97204, US

(21) Appl. No.: 10/435,604

(22) Filed: May 8, 2003

Related U.S. Application Data
(63) Continuation-in-part of application No. PCT/JP01/08371, filed on Sep. 26, 2001.

(30) Foreign Application Priority Data
Nov. 9, 2000 (JP) 2000-342623

(51) Int. Cl. A61K 48/00; C12Q 1/68

(52) U.S. Cl. 435/6; 514/44

(57) ABSTRACT

Efficient screening of genes that influence pathological conditions or survival of animals infected with pathogen is enabled by the use of a full-length cDNA library as DNAs for immunizing animals in place of a genomic DNA library, used in the conventional ELI method.

### CROSS REFERENCE TO RELATED APPLICATIONS

This application is a continuation-in-part of PCT/JP01/08371 filed Sep. 26, 2001, and claims priority from Japanese Application No. 2000-342623, filed Nov. 9, 2000.

### TECHNICAL FIELD

The present invention relates to a method of screening for genes that influence pathological conditions or survival of animals infected with pathogen. The present invention principally pertains to the field of pharmaceutical development.

### BACKGROUND ART

To date, the ELI (expression library immunization) method has been reported as a method of screening for genes that influence pathological conditions or survival of animals infected with pathogen (Michael A. B. et al., Nature 377, 632-635, 1995). According to this method, effective clones can be identified through the introduction of a genomic library into expression vectors, immunization of mice with the vectors followed by challenge infection, and monitoring of their influence on pathological conditions and survival of the mice.

In this method, however, the genomic library used as the target in the screening for genes of interest does not always contain parts of genes in frame. Therefore, the in vivo expression efficiency is low, which has been the major factor that decreases the efficiency of screening for genes of interest using the ELI method.

### DISCLOSURE OF THE INVENTION

The present invention was made in view of such situation, and an objective of the present invention is to provide a novel method of screening for genes that influence pathological conditions or survival of animals infected with pathogen, wherein the genes are screened with higher efficiency than the conventional ELI method.

To accomplish the objective, the inventors conducted extensive research and contemplated that the use of a full-length cDNA library in place of a genomic library, which is used in the conventional ELI method, as the DNAs for immunizing animals, would enable efficient screening of genes of interest. The inventors considered that a full-length cDNA library would be advantageous for in vivo gene expression, as opposed to a genomic library that does not always contain part of genes in frame due to the fact that the full-length cDNA library is generated from mRNA molecules corresponding to expressed genes and contains complete sequences of genes coding proteins.

Based on this idea, using a full-length cDNA library, the present inventors screened for genes that influence pathological conditions or survival of animals infected with a pathogenic eukaryotic organism. More specifically, a full-length cDNA library was constructed from Plasmodium berghei ANKA, a lethal strain of rodent malaria parasite, and mice were immunized to 2,000 clones randomly selected from this library. Subsequently, malaria parasites were infected to the mice to investigate the infection rate, pathological conditions and survival period after the infection of the mice. As a result, no significant difference in the parasite infection rate was observed between the group wherein the full-length cDNA library was administered and the control group. However, the survival period after the infection of the mice was significantly reduced in the group administered with the full-length cDNA library. These results were consistent with the observation that the mice in the group administered with the full-length cDNA library exhibited systemic piloerection, tremor, and convulsion. These results indicate that the full-length cDNA library administered to mice contains gene(s) that adversely affects the mice infected with pathogens.

Thus, the inventors have developed an ELI method that uses a full-length cDNA library as the immunogen, and found out that the use of this method enables screening of genes that influence pathological conditions or survival of animals infected with pathogen.

More specifically, the present invention provides:

(1) A method of screening for genes that influence pathological conditions or survival of vertebrates infected with pathogen, which method comprises the steps of:

(a) administering a full-length cDNA to a vertebrate;

(b) administering a pathogen to the vertebrate; and

(c) detecting changes in the pathological conditions or survival of the vertebrate after pathogen challenge, comparing with a control, and selecting the full-length cDNA that deteriorates or ameliorates the pathological conditions or survival of the vertebrate;

(2) The method according to (1), wherein the full-length cDNA is derived from pathogen;

(3) The method according to (1) or (2), wherein the pathogen is malaria parasite; and

(4) A gene isolated by the method according to any one of (1) to (3), which influences pathological conditions or survival of vertebrates infected with the pathogen.

(5) A DNA vaccine containing a plurality of full-length cDNA clones derived from pathogen;

(6) The DNA vaccine according to (5), wherein the pathogen is pathogenic protozoan;

(7) The DNA vaccine according to (6), wherein the pathogenic protozoan is Plasmodium berghei;

(8) The DNA vaccine according to any one of (5) to (7), wherein the plurality of full-length cDNA clones are 2,000 or more full-length cDNA clones;

(9) A method for enhancing immunity against a pathogen by administering to a vertebrate a plurality of full-length cDNA clones derived from the pathogen;

(10) The method according to (9), wherein the plurality of full-length cDNA clones are arbitrarily selected full-length cDNA clones;

(11) The method according to (9) or (10), wherein the pathogen is pathogenic protozoan;

(12) The method according to (11), wherein the pathogenic protozoan is Plasmodium berghei; and

(13) The method according to any one of (9) to (12), wherein the plurality of full-length cDNA clones are 2,000 or more full-length cDNA clones.

The present invention provides a method of screening for genes that influence pathological conditions or survival of vertebrates infected with pathogen. In the method of the present invention, first, full-length cDNAs are administered to vertebrates (step (a)).

Herein, there is no particular limitation on the full-length cDNAs administered to vertebrates. Any desirable full-length cDNAs that are expected to affect pathological conditions or survival of vertebrates infected with pathogen may be used. For the purpose of screening for DNA vaccines against pathogens, full-length cDNAs derived from the pathogens are preferably used. Plural kinds of full-length cDNAs (a full-length cDNA library) or one kind of a full-length cDNA alone may be administered to a vertebrate.

A full-length cDNA used in the step of the present invention may be prepared from various organisms according to methods described in the literature (Maruyama, K., Sugano, S., Gene 138, 171-174, 1994; Yutaka S. et al., Gene 200, 149-156, 1997). A messenger RNA of an eukaryote has a peculiar structure, which is called cap structure, at its 5′ end. According to the method described in the foregoing reference, tobacco acid pyrophosphatase that specifically recognizes the cap structure is used to replace the cap structure with a synthetic oligo-RNA, and then cDNA is generated using appropriate primers and reverse transcriptase. Specifically, messenger RNA extracted and purified from an organism is pretreated with bacterial alkaline phosphatase to remove 5′-phosphates of non-capped mRNAs. Subsequently, the RNA molecules from which 5′-phosphates have been removed are treated with tobacco acid pyrophosphatase to cleave the phosphate bonds in the cap structure. As a result, a single phosphate will be left at the 5′ end of the RNA molecules. Then, a synthetic oligo-RNA molecule is ligated to the 5′ end of the RNA molecules by the action of RNA ligase. Thus, synthetic oligo-RNA molecules are selectively attached to the 5′ end of the full-length messenger RNAs having the cap structure. Then, cDNAs are synthesized with poly(T) primers and RNase H free-reverse transcriptase using the obtained RNA molecules as templates to obtain full-length cDNAs.

Non-human vertebrates, including mammals and aves, may be used as subjects to administer the full-length cDNAs of the present invention. Preferably, the non-human vertebrate is a mammal. Among mammals, rodents, such as mice, are particularly preferred for their low cost, ease of breeding in large numbers, and facility to conduct experiments with large number of animals. For application to humans, primates such as monkeys are preferred.

Full-length cDNA may be integrated into a vector that ensures in vivo expression of the cDNA, and then administered to animals by means of, for example, intraspleen, injection, intraperitoneal injection, intramuscular injection, subcutaneous injection, percutaneous injection, intrapleural inoculation, intracerebral inoculation, gene gun, oral inoculation, intranasal inoculation, and inhalation through the respiratory tract. The dosage should be an amount that is expected to cause a sufficient effect but that does not cause toxicity or side effect. One skilled in the art can properly determine the optimum dosage. Typically, a dose of 0.1 μg to 100 μg is administered at a time and, if necessary, multiple administration may be conducted.

In the method of the present invention, next, a pathogen is administered to the vertebrate that had been administered with the full-length cDNA (step (b)).

There is no particular limitation on the pathogens that are administered to animals so long as they are pathogenic to the animals. Such pathogens include pathogenic protozoa, pathogenic fungi, and pathogenic eukaryotic microorganisms. Administration of pathogens to animals may be conducted via, for example, intraperitoneal, intravenous, subcutaneous, and intramuscular injections, inoculation by insect vectors, intranasal administration, aerial infection, etc. An amount of pathogen that is required for exhibiting pathogenicity is administered to an animal. One skilled in the art can properly decide such dose of a particular pathogen.

Next, in the method of the present invention, the changes in pathological conditions or survival of vertebrates after the pathogen challenge are detected and compared with that of the control, and the full-length cDNA that deteriorates or ameliorates the pathological conditions or survival of the vertebrates is selected (step (c)).

As used herein, the phrase “changes in pathological conditions” refers to various changes in pathological conditions caused in animals infected with pathogen. Pathological conditions may vary depending on pathogens that are infected to animals, and include, for example, weight loss, anemia, and psychotic manifestations. “Changes in survival” refers to changes in the survival period or survival rate of the animals infected with pathogen.

In this step of the present invention, an empty vector that contains no insert of full-length cDNA may be used as a control for determining the influence of the full-length cDNA on pathological conditions or survival. When deterioration of pathological conditions or survival is observed for the group administered with a full-length cDNA compared to the control group through the detection of changes in the pathological conditions or survival, the full-length cDNA is determined to encode a polypeptide that adversely affects the pathological conditions or survival of the vertebrates infected with the pathogen. On the contrary, when amelioration of pathological conditions or survival is observed for the group administered with a full-length cDNA compared to the control group, the full-length cDNA is determined to encode a polypeptide that affects the pathological conditions or survival of the vertebrates infected with the pathogen in a beneficial way.

Genes identified by the method of the present invention that adversely affect the host animals are suggested to be closely associated with the pathogenicity of diseases. Thus, such genes are not only important to elucidate the mechanism of pathogenicity of a pathogen to a host animal, but also for conducting researches aiming to reduce pathogenicity, such as development of pharmaceuticals wherein identified genes or proteins encoded by the genes are used as targets. On the other hand, genes identified by the method of the present invention that affect the host animals in a beneficial way are considered to be useful, for example, for gene therapy of pathogen infections and as DNA vaccines to prevent pathogen infections.

Furthermore, the present invention provides a DNA vaccine containing plurality of full-length cDNA clones derived from pathogen. The term “DNA vaccine” herein refers to a vaccine wherein DNA is inoculated as the immunogen. An immune response similar to that caused by natural infection of a pathogen is suggested to be induced due to antigenic proteins which are synthesized upon introduction of a DNA encoding a pathogenic antigen.

The above-mentioned pathogen may be, for example, those described above, but preferably it is a pathogenic protozoan, and more specific examples include Plasmodium berghei.

In the present invention, a cDNA clone can be defined as “a homogeneous group of full-length cDNAs having essentially the same nucleotide sequence”.

Herein, the phrase “plurality of full-length cDNA clones” refers usually to 100 or more, preferably 1,000 or more, and especially preferably 2,000 or more full-length cDNA clones. Furthermore, there are no particular limitations on the full-length cDNA clones, but arbitrarily selected full-length clones are preferable.

Furthermore, the present invention relates to a method of enhancing immunity against a pathogen by administering full-length cDNA clones derived from the pathogen to vertebrates. In a preferred embodiment of the above-mentioned method, the “plurality of full-length cDNA clones” are arbitrarily selected full-length cDNA clones.

In the above-mentioned method, examples of the target for administering the “plurality of full-length cDNA clones” include vertebrates, such as mammals (including humans) and aves, and mammals are preferred.

The method of administration in the above-mentioned method is already described above.

### BRIEF DESCRIPTION OF THE DRAWINGS

FIG. 1 shows the survival rates over the course of time after the protozoan infection of mice administered with the full-length cDNA libraries.

FIG. 2 shows the protozoan infection rates of mice administered the full-length cDNA libraries over the course of time.

FIG. 3 shows the survival rates of mice administered with plurality of full-length cDNA clones over the course of time after the protozoan infection.

FIG. 4 shows the protozoan infection rates of mice administered with plurality of full-length cDNA clones over the course of time.

FIG. 5 shows the protozoan infection rates of mice administered with the empty control vector over the course of time.

### BEST MODE FOR CARRYING OUT THE INVENTION

Herein below, the present invention will be specifically described with reference to Examples, however, it is not to be construed as being limited thereto.

### EXAMPLE 1

### Construction of Full-Length cDNA Library

A strain of murine malaria parasite, Plasmodium berghei ANKA, was inoculated into Wistar rat, and protozoan parasites were proliferated in the rat. After anesthetizing the rat, blood was collected from the heart into a syringe containing EDTA. Whole blood was filtered through a Plasmodipur filter to remove leukocytes, and collected by centrifugation. Twenty volumes of Trizol LS was added to the infected erythrocytes, and the protozoan parasites were homogenized by pipetting. Total RNA was then collected according to the protocol of Trizol LS, and poly-A RNA was purified using oligo-tex column. Full-length cDNA was prepared from the poly-A RNA according to the method described in the literature (Maruyama, K., Sugano, S., Gene 138: 171-174, 1994; Yutaka S. et al., Gene 200: 149-156, 1997). The prepared cDNA was introduced into pCE-FL vector. The pCE-FL was prepared from pME18S-FL3 (Accession No. AB009864) and has an EF321 promoter and CMV-IE enhancer located upstream of the insert.

### EXAMPLE 2

### Influence of Immunization With Full-Length cDNA Libraries on Mice Infected With Protozoan Parasites

2,000 clones were randomly selected from the constructed library to increase the clones in E. coli. Plasmid DNA was purified from the E. coli using QIAGEN Endotoxin-free kit (QIAGEN plasmid purification kit). Five sets of sub-libraries were similarly prepared. As a control, an empty vector carrying no insert was prepared in the same way to be used for immunization. 50 μg of the DNA was dissolved in 50 μl of physiological saline, and directly injected into the spleens of BALB/c mice. Specifically, a small incision was made in the lateral abdomen of each mouse under anesthetization, the spleen was taken out, injection was performed under visualization, the spleen was put back into the peritoneal cavity, and the incision was sewed. After one week, the same amount of the DNA was injected into the muscle. This injection was repeated after one week in the same way. One week after the third immunization, 50,000 erythrocytes infected with Plasmodium berghei ANKA were inoculated into the peritoneal cavity. After the inoculation, the infected mice were daily examined and weighed. Blood samples were taken from the tail of each mouse every second day to prepare blood smear specimens, which were subjected to Giemsa staining to determine the protozoan infection rate.

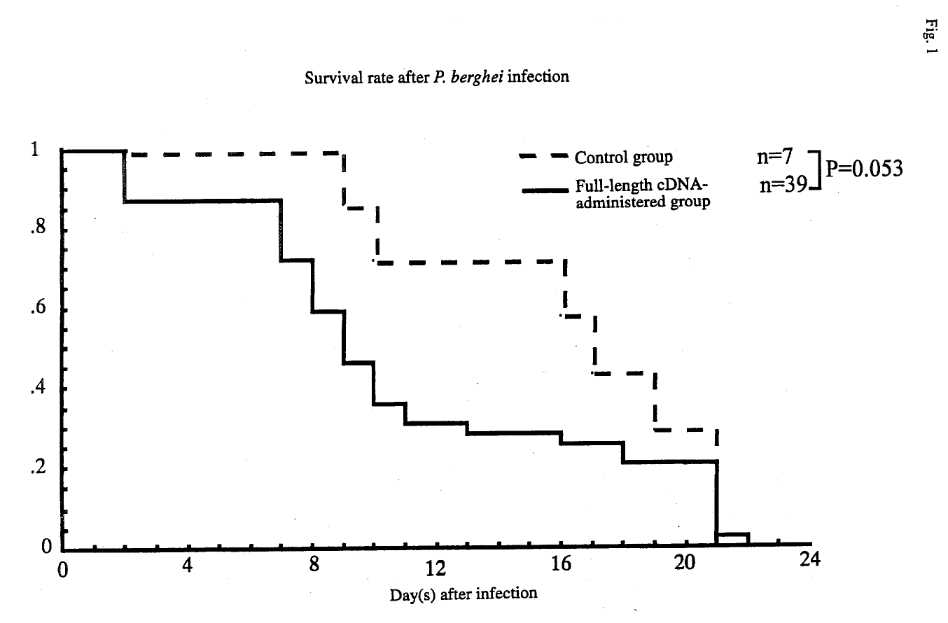

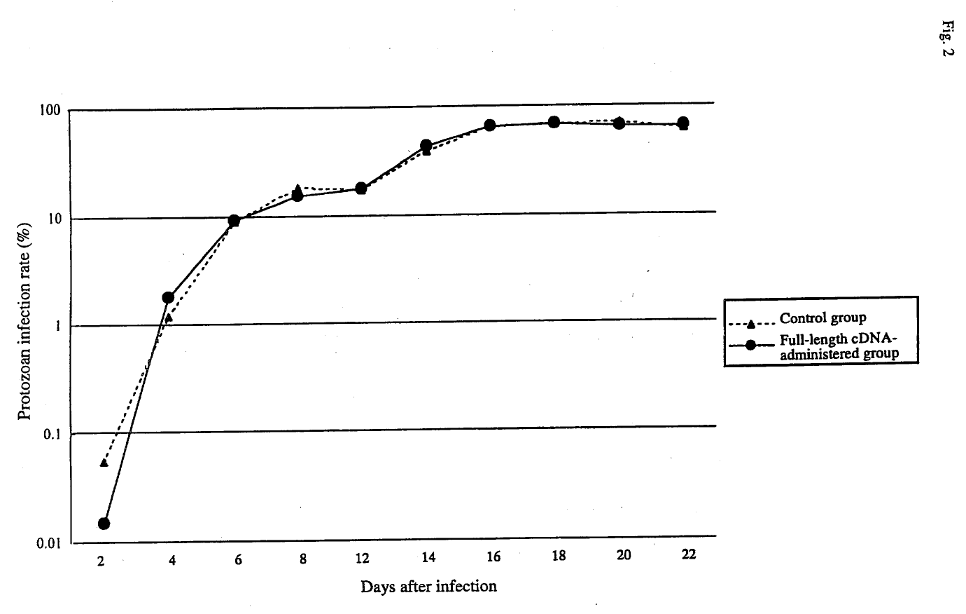

Compared to the control, the survival period was reduced in the group administered with the full-length cDNA library that was constructed from murine malaria parasite (FIG. 1). Comparison with the control group by taking the five groups administered with the full-length cDNA libraries as one group using the Kaplan-Meier method revealed a P-value of 0.053. No significant difference between the two groups was observed for the changes in the body weight and protozoan infection rates (FIG. 2).

In addition, sequencing of a part of the expression vector library used for immunization revealed that the library contained malaria protozoan genes, host rat genes, and chimeric genes of malaria protozoa and rats. Thus, it was suggested that any one of these genes or multiple genes thereof in combination may affect the survival period.

### EXAMPLE 3

### Preparation of Immunological Material

A colony of 2,000 clones, group G2000, was stocked as one batch from a full-length cDNA library prepared from the Plasmodium berghei ANKA strain of erythrocytic protozoa. Using the colony as a seed, E. coli was cultured as one batch, and then plasmid DNA was purified using Endotoxin-free plasmid preparation kit (QIAGEN). Using the plasmid DNA as the material, immunological gold particles for Bio-Rad Gene gun were prepared. An empty vector without the insert was prepared in the same way to produce gold particles as a control.

The experiment was performed using eight 5-week old female BALB/c mice as one group. The abdomen of each mouse was shaved using shaving cream, and immunized at 5 positions, left and right upper abdomen, umbilical region, and left and right inguinal region, using Bio-Rad Gene gun. The amount of administered DNA was 1 microgram per shot, and 5 shots (5 microgram) for one immunization per animal. The control group was immunized similarly. Immunization was repeated 3 times ever week. One week after the final immunization, 50,000 erythrocytes infected with P. berghei ANKA protozoan were suspended in 0.1 mL of physiological saline, then infection was performed by intraperitonial inoculation of the erythrocytes. Subsequently, daily observation and observation of protozoan infection rate every other day were continued until the death of all animals.

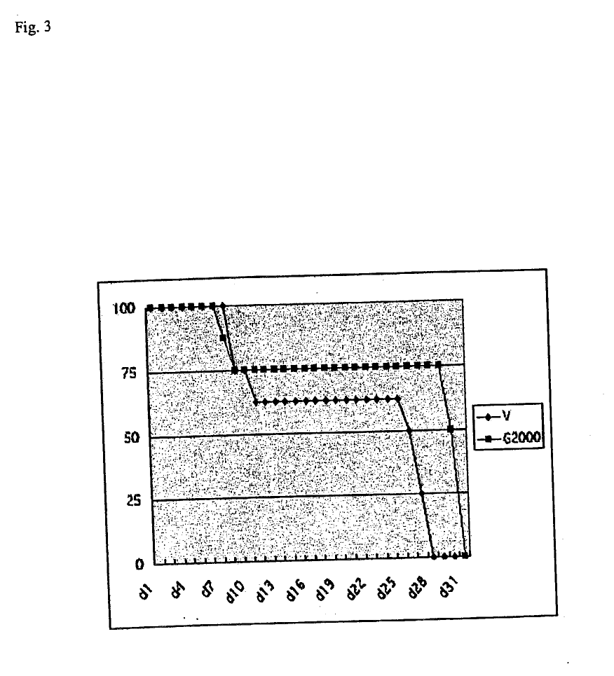

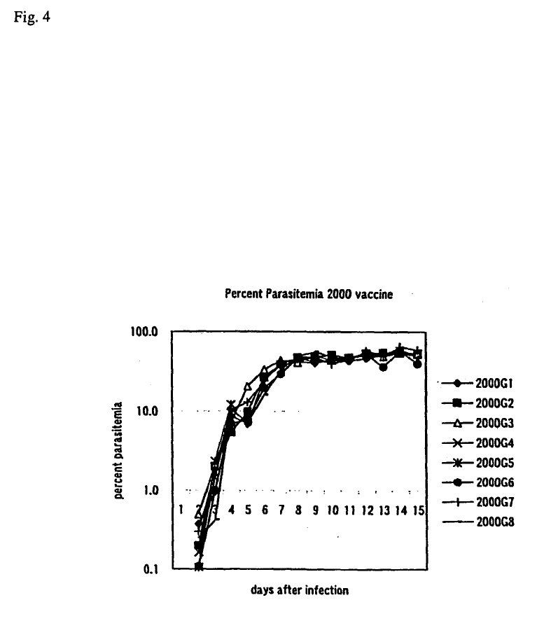

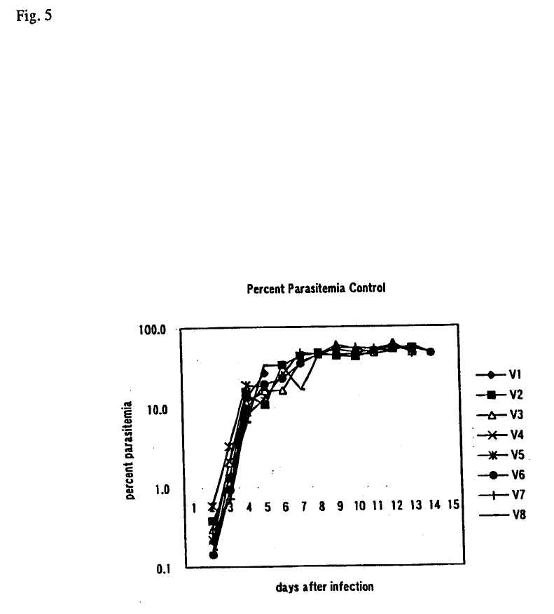

Survival period was analyzed by Cox's F-test of Kaplan-Meier. Survival of the 2,000 clone immunized group was extended with a significance of P=0.04 compared to the control vaccine (empty vector) immunized group (FIGS. 3-5).

According to the above results, immunization of a plurality of clones as a DNA vaccine was suggested to elongate the survival of animals. Furthermore, development of effective vaccine by immunological administration of plural kinds of protozoan-derived proteins can be expected from these results.

### INDUSTRIAL APPLICABILITY

The present invention enables efficient screening of genes that influence pathological conditions or survival of animals infected with pathogen. The method of the present invention contributes to the development of therapeutics and prophylactics against pathogens, as well as to the elucidation of the pathogenicity of a pathogen against a host animal.

I claim:

1. A method of screening for genes that influence pathological conditions or survival of vertebrates infected with pathogen comprising:
administering a full-length cDNA to a vertebrate;
administering a pathogen to the vertebrate; and
detecting changes in the pathological conditions or survival of the vertebrate after pathogen challenge, comparing with a control, and selecting the full-length cDNA that deteriorates or ameliorates the pathological conditions or survival of the vertebrate.

2. The method according to claim 1, wherein the full-length cDNA is derived from pathogen.

3. The method according to claim 1 or 2, wherein the pathogen is a malaria parasite.

4. A gene isolated by the method of claim 1, wherein the gene influences pathological conditions or survival of vertebrates infected with the pathogen.

5. A DNA vaccine containing a plurality of full-length cDNA clones derived from pathogen.

6. The DNA vaccine according to claim 5, wherein the pathogen is pathogenic protozoan.

7. The DNA vaccine according to claim 6, wherein the pathogenic protozoan is Plasmodium berghei.

8. The DNA vaccine according to anyone of claims 5 to 7, wherein the plurality of full-length cDNA clones are 2,000 or more full-length cDNA clones.

9. A method for enhancing immunity against a pathogen by administering to a vertebrate a plurality of full-length cDNA clones derived from the pathogen.

10. The method according to claim 9, wherein the plurality of full-length cDNA clones are arbitrarily selected full-length cDNA clones.

11. The method according to claim 9, wherein the pathogen is pathogenic protozoan.

12. The method according to claim 11, wherein the pathogenic protozoan is Plasmodium berghei.

13. The method according to any one of claims 9 to 12, wherein the plurality of full-length cDNA clones are 2,000 or more full-length cDNA clones.
